(19) United States
(12) Patent Application Publication
(10) Pub. No.: US 2018/0140410 A1
(43) Pub. Date: May 24, 2018

(54) COSMETIC IMPLANT

(71) Applicant: William A. Brennan, Lafayette, LA (US)

(72) Inventors: William A. Brennan, Lafayette, LA (US); Kevin Yacoub, Los Olivos, CA (US)

(21) Appl. No.: 15/814,523

(22) Filed: Nov. 16, 2017

Related U.S. Application Data
(60) Provisional application No. 62/424,590, filed on Nov. 21, 2016.

(51) Int. Cl. A61F 2/00; A61F 2/12

(52) U.S. Cl. CPC A61F 2/0059; A61L 27/52; A61F 2/12

(57) ABSTRACT

A cosmetic implant includes a silicone gel core and an outermost hydrophilic coating on all sides of the silicone gel core. An intermediate coating can be provided between the silicone gel core and the outermost coating. A method of performing cosmetic surgery on a patient is also disclosed.

### CROSS-REFERENCE TO RELATED APPLICATIONS

This application claims priority to U.S. Provisional Patent Application No. 62/424,590 filed Nov. 21, 2016, entitled “Cosmetic Implant”, the entirety of which is incorporated herein by reference.

### FIELD OF THE INVENTION

This invention relates generally to medical implants, and more particularly to cosmetic implants.

### BACKGROUND OF THE INVENTION

Cosmetic augmentation has been performed with synthetic devices for over 50 years. Devices designed to be surgically implanted into human tissue have been developed and used in many human tissue areas such as breast, buttocks, calf, pectoral and others. The development of these implant devices over the years has ranged from liquid filled to solid substances and many include silicone based components. More recently silicone gel filled implants have become popular because of their resistance to rupture and widespread filling material extravasation as well as their more natural appearance and texture. The vast majority of breast implants is a single large implant in the form of a silicone shell that is filled with a saline solution or silicone gel filling material. A significant complication that can occur with the single large implant is capsular contracture, where abnormal scar tissue forms around the implant.

The field of cosmetic human tissue augmentation has not enjoyed the same advantages of minimally invasive technology as other surgical science fields and developments of a minimally invasive cosmetic augmentation approach have gained only limited attention. A major disadvantage of microimplants proposed in the past is the propensity for these microimplants to develop high friction between each other leading to shear forces between the microimplants as well as creating a texture to external palpation of the human tissue. Microballoons in the past have been described as a polymeric shell with a filling substance. The requirement of a filling port makes a smooth and continuous outer surface design impossible. Also, typical pendant functionality in cured silicone compositions is trimethyl. This creates a very high surface friction in typical silicones. This silicone to silicone coefficient of friction is >1.0.

Systems and methods for breast augmentation are shown in U.S. Pat. No. 7,169,180 issued Jan. 30, 2007 and U.S. Pat. No. 8,092,527 issued Jan. 10, 2012. The disclosures of these references are hereby incorporated fully by reference. These systems describe microballoons including a flexible, enclosed shell defining an open interior that is filled with a liquid, gas or gel filling material.

### BRIEF DESCRIPTION OF THE DRAWINGS

There are shown in the drawings embodiments that are presently preferred it being understood that the invention is not limited to the arrangements and instrumentalities shown, wherein:

FIG. 1 is a schematic depiction of a cosmetic implant according to the invention.

### SUMMARY OF THE INVENTION

A cosmetic implant includes a silicone gel core and an outermost hydrophilic coating on all sides of the silicone gel core.

The silicone gel core can have a gel penetration stiffness of between 2.5 and 20 mm. The silicone gel core can have a gel penetration stiffness between 5.0 and 15 mm. The silicone gel core can have a gel penetration stiffness between 8.5 and 10.5 mm.

The elastic modulus of the silicone gel core can be between 1,000 and 15,000 Pascals. The elastic modulus of the silicone gel core can be between 2,000 and 10,000 Pascals. The elastic modulus of the silicone gel core can be between 3,000 and 9,500 Pascals.

The diameter of the silicone gel core can be from 0.5 to 15 mm. The diameter of the silicone gel core is from 1 to 12 mm. The diameter of the silicone gel core can be from 3 to 10 mm.

The outermost coating can have a resistance force between 0.1 and 30 grams. The outermost coating can have a resistance force of between 1 and 20 grams. The outermost coating can have a resistance force of between 2 and 15 grams.

The outermost coating can have a thickness of between 5 μm and 190 μm.

The outermost coating can comprise at least one selected from the group consisting of polyvinylpyrrolidone, hyaluronic acid, polylactic acid, polyethylene glycol, collagen and chitosan.

The silicone gel core can be formed from a reactive polydimethyl siloxane polymer having a viscosity of from 100 centipoises to 100,000 centipoises. The silicone gel core can comprise at least one selected from the group consisting of Nusil MED-6342, Nusil MED-6345, Nusil MED-6350, Nusil MED-6311, Applied Silicone 40022, Applied Silicone 40135, and Applied Silicone 40008.

The cosmetic implant can include an intermediate coating between the silicone gel core and the outermost coating. The intermediate coating can comprise a resinous silicone coating on all sides of the silicone gel core; wherein the intermediate coating is between and adhered to both of the silicone gel core and to the outermost coating. The intermediate coating can have a thickness of from 0.5 to 500 microns.

The outermost coating can have a implant to implant static and kinetic coefficient of friction between 0.025 and 1.0. The outermost coating can provide an implant to implant static and kinetic coefficient of friction between 0.05 and 0.6. The outermost coating can provide a implant to implant static and kinetic coefficient of friction between 0.1 and 0.4.

The intermediate coating can have a static and kinetic coefficient of friction between 0.025 and 1.0.

A method of making a cosmetic implant can include the steps of forming a silicone gel core and coating the silicone gel core with an outermost hydrophilic coating layer. An intermediate resinous silicone coating layer can be provided on all sides of the silicone gel core, and the intermediate coating layer can be coated with the outermost continuous hydrophilic coating on all sides of the intermediate coating layer. The intermediate coating layer is between and adhered to both of the silicone gel core and the outermost hydrophilic coating layer.

A method of performing cosmetic surgery on a patient, includes the step of providing a plurality of cosmetic implants, the implants comprising a silicone gel core and an outermost continuous hydrophilic coating on all sides of the silicone gel core. An incision is made in the patient to provide access to a subcutaneous pocket. A plurality of cosmetic implants are placed in the subcutaneous pocket. The method can further include the step of forming a subcutaneous pocket after making the incision.

A cosmetic implant can include a biocompatible and deformable material with a hydrophilic outer surface which is in constant contact with either human tissue or two or more cosmetic implants of same or similar composition.

A cosmetic implant can include a silicone based deformable material with a hydrophilic outer surface which is in constant contact with either human tissue or two or more cosmetic implants of same or similar composition.

A cosmetic implant can include a silicone gel with a hydrophilic outer surface which is either in constant contact with either human tissue or two or more cosmetic implants of same or similar composition.

### DETAILED DESCRIPTION OF THE INVENTION

Breast augmentation requires supplementing the human tissue with prosthetic device(s). The minimally invasive method of the invention begins with a small incision, through which the surgeon develops a tissue pocket under the desired tissue for augmentation. The surgeon then delivers three or more deformable microimplants into the tissue pocket to achieve tissue augmentation. The microimplants are constructed of biologically compatible material, deformable in character and possess an external hydrophilic surface to reduce friction between the microimplants as well as reduce friction between the implants and the host human tissue. The microimplants do not require a filling and thus do not require a filling port. The microimplants of the invention are composed of a semisolid elastomeric core made from a material which provides some give to external forces such as palpation, but is not rigid to the touch and does not flow or deform without external force at body temperatures. A lubricious hydrophilic material coats the semisolid elastomeric core. The coating facilitates the movement of the implants relative to one another and to surrounding tissue forming the subcutaneous pocket. This movement improves both the appearance and feel of the implant. In addition the exterior hydrophillic outer surface having pendant —OH functionality or similar hydrophilic functionality will provide a much more biocompatible surface for the implant. This is possible from the surface chemistry mimics the aqueous interior of our body tissues and fluids.

A cosmetic implant according to the invention can include a biocompatible and deformable material with a hydrophilic surface which is in constant contact with either human tissue or two or more cosmetic implants of the same or similar composition. The cosmetic implant can comprise a silicone based deformable material with a hydrophilic outer coating which is in constant contact with either human tissue or two or more cosmetic implants of same or similar composition. The coating can cover all of the outer surface. The cosmetic implant can more particularly comprise a silicone gel with a hydrophilic outer coating which is in constant contact with either human tissue or two or more cosmetic implants of same or similar composition.

The dimension of the microimplants can vary. In the preferred embodiment the diameter or largest dimension of the microimplants is between 3 mm and 15 mm in diameter. The diameter or largest dimension of the microimplants can be between 3 mm and 10 mm. In another embodiment the diameter is 1 mm to 12 mm. In another embodiment the diameter is 0.5 mm to 15 mm. The diameter or largest dimension of the implants can be within a range of any high and low value selected from 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, and 15 mm.

The microimplants can have any suitable shape. In one embodiment the microimplants have a spherical shape. The spherical shape provides a reduced area of contact between microimplants and helps to prevent or reduce implant-implant interaction. Other shapes are possible.

The elastomeric core can be produced by any suitable technique Silicone gels are formulated to have very low crosslink density. These low crosslink density gels are very soft and deformable by compression. However, as lightly crosslinked rubbers, they return to their original shape after being compressed. Many formulation techniques can result in gels. These include the use of chain extenders, very high molecular weight polymers, very high molecular weight crosslinkers, as well the use of a starved crosslink condition will result in a silicone gel. A starved crosslinker condition is where a significantly reduced stoichiometrically level of crosslinker is utilized to create a very low crosslink density rubber/gel. In the preferred embodiment the elastomeric core comprises a silicone based gel that can be produced via either injection molding or compression molding. Other methods of forming the core are possible.

The elastomeric semisolid core can have a stiffness that is between 8.5 and 10.5 mm as measured by penetration using a Labline penetrometer equipped with a ¼″ probe that weighs 57.3 grams. The penetration is measured by measuring the distance that the ¼″ probe descends into the elastomeric core after 5 seconds under the force of gravity. In another embodiment the stiffness can be between 5.0 mm and 15 mm, and in yet another embodiment the stiffness can be between 2.5 and 20 mm. The stiffness can be within a range of any high and low value selected from 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5 and 20.0 mm.

[30] The elastic modulus of the gel core can be between 3000 and 9500 Pascals when measured using an TA Instruments (New Castle Del.) AR2000 or Ares rotational rheometer equipped with 25 mm parallel plates. The rheometry test is performed by applying the uncured silicone gel between the 40 mm diameter plates. The distance between the plates is 0.5 mm. The test is initiated at room temperature and then heated to 150° C. at a ramp rate of 7.5° C./minute. The oscillatory strain of the test is 3.3% with a frequency of 1 Hz. In another embodiment, the elastic modulus can be between 2000 and 10,000 Pascals, and in yet another embodiment the elastic modulus can be between 1000 and 15,000 Pascals. In another embodiment, the elastic modulus can be within a range of any high and low value selected from 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, and 15000 Pascals.

The elastomeric core can be a silicone-based gel. The term “silicone-based” as used herein means a reactive polydimethyl siloxane polymer with a viscosity of 100 centipoise to 100,000 centipoise but preferably about 1000 centipoise. The silicone-based gel is supplied as a 2-part system where the Part A contains the vinyl-endblocked dimethyl siloxane polymer and platinum catalyst. The Part B contains the vinyl endblocked dimethyl siloxane polymer, methyl-hydrogen crosslinker, and a suitable inhibitor such as methylvinylcyclosiloxane. The reactive siloxane polymer can have terminal vinyl groups pendant vinyl groups, or a combination of both. The siloxane polymer should have dimethyl substituted groups along the backbone but can also have diphenyl, methylphenyl, and trifluoropropyl substitution. The crosslinker can possess terminal hydride groups, pendant hydride groups, or a combination of both. The hydride concentration can range from 10 to 80 mole % but preferably 50 mole %. The catalyst should be platinum based at a concentration of 2 to 10 parts per million but preferably 8 ppm. Other catalysts can be iridium, palladium, rhodium, and other suitable catalysts. Examples of suitable materials include Nusil MED-6342, Nusil MED-6345, Nusil MED-6350, Nusil MED-6311 (NuSil Technology LLC, Carpinteria, Calif.) and Applied Silicone 40022, Applied Silicone 40135, and Applied Silicone 40008 (Applied Silicone Corporation, Santa Paula, Calif.).

In order to allow this device to interact with human tissue and other microimplants with as little friction as possible, a hydrophilic outermost coating layer is applied over the gel core. The outermost coating comprises a material that adheres to the elastomeric semisolid core or any coating between the elastomeric core and the outer coating, such as an intermediate coating layer to be described below, and also provides a hydrophilic property so as to produce a lubricious quality to the coating as it contacts human tissue as well as other microimplants. In the preferred embodiment the resistance force of the outermost lubricious coating can be between 2 grams and 15 grams using industry standard methods as described below. In another embodiment the resistance force can be between 1 gram and 20 grams and in yet another embodiment it can be between 0.1 grams and 30 grams. In another embodiment, the resistance force can be within a range of any high and low value selected from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 grams.

The resistance force is measured by pulling a coated sample between two silicone rubber pads. The coated sample is clamped between the two rubber pads with 500 grams of clamping force and the coated sample is pulled through the rubber pads. A load cell is attached to the test fixture and measures the force as the coated sample is pulled through the rubber pads.

Typical trimethyl terminated, hydrophobic silicone to silicone have a high >1.0 coefficient of friction. By comparison, PTFE, Teflon has a coefficient of friction of 0.05 to 0.10 and steel has a coefficient of friction of 0.6. In contrast to typical silicone high >1.0 coefficient of friction, in the invention the implant to implant static and kinetic coefficient of friction of the outermost coating can be between 0.1 and 0.4. In another embodiment the implant to implant static and kinetic coefficient of friction can be between 0.05 and 0.6. In yet another embodiment the implant to implant static and kinetic coefficient of friction can be between 0.025 and 1.0. In another embodiment, the implant to implant static and kinetic coefficient of friction can be within a range of any high and low value selected from 0.025, 0.05, 0.075, 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, and 1.

The implant to implant static and kinetic coefficient of friction was measured using a standard coefficient of friction tester such as the Thwing-Albert FP-2260 COF Tester (Thwing-Albert Instrument Company, West Berlin N.J.). The test is conducted in accordance with ASTM D1894 by coating a piece of cured silicone elastomer with the resinous coating. The coefficient of friction is measured by sliding a metal block referred to as a sled, along the coated test sample. Static friction applies to the force necessary to initialize motion between the two surfaces and kinetic friction is the resistance to sliding once the surfaces are in relative motion.

The hydrophilic outermost coating layer can be comprised of a hydrogel which is a crosslinked hydrophilic polymer that swells in the presence of water to provide lubricity. The hydrophilic polymer for the outermost coating layer can be synthetic such as polyvinylpyrrolidone or natural such as collagen and chitosan. The hydrophilic polymer coating can be crosslinked with heat, UV, plasma, or corona to create a solid outer bonded surface. Suitable materials for the hydrophilic hydrogel outer layer include polyvinylpyrrolidone, hyaluronic acid, polylactic acid, and polyethylene glycol. Examples of suitable materials include Harland Medical Systems Lubricent (Harland Medical Systems, Inc., Eden Prairie, Minn.), Surmodics Serene lubricious coating (Surmodics, Inc., Eden Prairie, Minn.), AST Products Lubrilast lubricious coating (AST Products, Inc., Billerica Mass.), ISureTec Isureglide lubricious coating (Biomedical Inc. St. Paul, Minn.), and DSM Comfortcoat lubricious coating (DSM Biomedical, Inc., Exton, Pa.). The term “hydrophilic” as used herein refers to a compound which is attracted to or absorbs water. Another benefit of the outermost hydrophilic coating layer in addition to lubricity is to create an outer aqueous environment for the microsphere. This creates a more biocompatable surface chemistry. Hydrogen bonding water will surround the particles help lubrication.

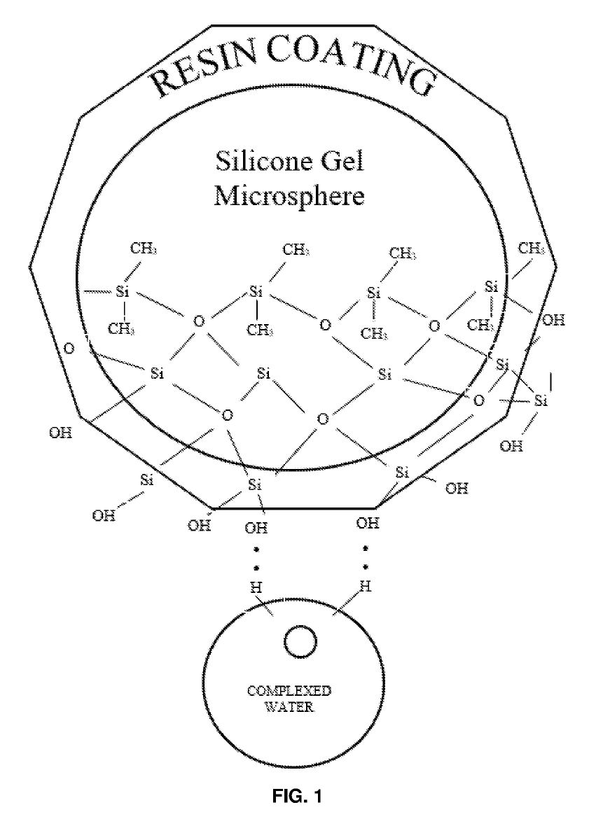

In order to counteract the tacky nature of the silicone gel core, an intermediate resinous silicone coating can be applied to reduce the interactive friction between the gel cores during production of the implants. The resinous coating is applied to the gel core to facilitate handling, and must adhere to the gel core and then also to the material forming the outermost hydrophilic layer that is applied over the intermediate layer. The intermediate layer can be applied by dipping or spraying onto the gel core and is cured using heat. Upon curing, the intermediate layer forms a resinous non-stick surface bonded to the outer surface of the gel core. The intermediate layer can be comprised of a trifunctional silane such as ethyltriacetoxysilane and condensation catalyst such as titanium butoxide which are diluted in a non-polar solvent such as xylene, toluene, hexane, or heptane. The trifunctional silane can also be methyltriacetoxysilane, vinyltrimethoxysilane, vinyltriethyoxysilane, and/or methyltriethoxysilane. An alternate condensation catalyst can be organotin or Titanium diisopropoxide bis(acetylacetonate). The coefficient of friction for the intermediate layer can be measured in the same manner as described above for the outer hydrophilic layer, and can have the same ranges of static and kinetic coefficients of friction. A cosmetic implant with a silicone gel core—intermediate layer—outermost layer construction is depicted schematically in FIG. 1. FIG. 1 illustrates three layers, the interior microspheres with a lubricious outer chemistry (silanol), the silicone shell which can be made of typical silicone rubber, and a the resin outer coating of the silicone shell depicting the hydrophillic surface for lubricity and bio-compatibility. Silanol (Si—OH) is used in this drawing, however many surface functionalities will enable the hydrophillic surface chemistry.

Suitable materials for the intermediate coating layer include NuSil MED-6670 (NuSil Technology LLC, Carpinteria Calif.). Another example of a suitable intermediate coating layer material is Momentive Silopren LSR Topcoat (Momentive Performance Materials Inc., Waterford, N.Y.).

The thickness of the intermediate silicone coating layer in the preferred embodiment is from 10 microns to 75 microns. In another embodiment the thickness of the intermediate silicone coating is 5 microns to 100 microns. In yet another embodiment the thickness of the intermediate silicone coating is 1 micron to 190 microns. In yet another embodiment the thickness of the silicone coating is 0.5 microns to 500 microns. The thickness of the intermediate silicone coating can be within a range of any high and low value selected from 0.5, 0.6, 0.7, 0.8, 0.9, 1, 2, 3, 4, 5, 10, 15, 20, 25, 30, 35, 40, 45, 50, 55, 60, 65, 70, 75, 80, 90, 100, 110, 120, 130, 140, 150, 160, 170, 180, 190, 200, 225, 250, 275, 300, 325, 350, 375, 400, 425, 450, 475 and 500 microns.

The thickness of the lubricious hydrophilic outer layer can be 25-50 μm but can be from 5 to 190 μm. The thickness of the lubricious hydrophilic outer layer can be within a range of any high and low value selected from 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180 and 190 μm.

The term coating layer or layer as used herein means that the layer forms a coating covering all sides of the surface that is being coated. There is no exposed portion or no substantial exposed portion of the underlying layer. The coating layer can coat 50%, 60%, 70%, 80%, 90%, 91%, 92%, 93%, 94%, 95%, 96%, 97%, 98%, 99%, 99.1%, 99.2% 99.3%, 99.4%, 99.5%, 99.6%, 99.7%, 99.8%, 99.9% or 100% of the surface area of the layer that is being coated or within a range of any high and low value selected from these values.

Once produced this microimplant can be delivered into a subcutaneous pocket in human tissue and serve to cosmetically enhance cosmetic areas such as breast, buttock, pectoral, calf and other areas. In the preferred embodiment, these microimplants are delivered into the subcutaneous pocket through a tube of similar diameter or smaller diameter using a plunger to push the microimplants into the subcutaneous pocket. The size and number of implants that are delivered to the area by the surgeon will depend on the area and the treatment plan. In one embodiment in the area of cosmetic augmentation of the breast, it is anticipated that between 3 and 3000, between 3 and 1000, or between 50 and 300 implants can be delivered to the surgical site.

This invention can be embodied in other forms without departing from the spirit or essential attributes thereof.

We claim:

1. A cosmetic implant, comprising:
a silicone gel core;
an outermost hydrophilic coating on all sides of the silicone gel core.

2. The cosmetic implant of claim 1, wherein the silicone gel core has a gel penetration stiffness of between 2.5 and 20 mm.

3. The cosmetic implant of claim 1, wherein the silicone gel core has a gel penetration stiffness between 5.0 and 15 mm.

4. The cosmetic implant of claim 1, wherein the silicone gel core has a gel penetration stiffness between 8.5 and 10.5 mm.

5. The cosmetic implant of claim 1, wherein the elastic modulus of the silicone gel core is between 1,000 and 15,000 Pascals.

6. The cosmetic implant of claim 1, wherein the elastic modulus of the silicone gel core is between 2,000 and 10,000 Pascals.

7. The cosmetic implant of claim 1, wherein the elastic modulus of the silicone gel core is between 3,000 and 9,500 Pascals.

8. The cosmetic implant of claim 1, wherein the diameter of the silicone gel core is from 0.5 to 15 mm.

9. The cosmetic implant of claim 1, wherein the diameter of the silicone gel core is from 1 to 12 mm.

10. The cosmetic implant of claim 1, wherein the diameter of the silicone gel core is from 3 to 10 mm.

11. The cosmetic implant of claim 1, wherein the outermost coating has a resistance force between 0.1 and 30 grams.

12. The cosmetic implant of claim 1, wherein the outermost coating has a resistance force of between 1 and 20 grams.

13. The cosmetic implant of claim 1 wherein the outermost coating has a resistance force of between 2 and 15 grams.

14. The cosmetic implant of claim 1, wherein the outermost coating has a thickness of between 5 μm and 190 μm.

15. The cosmetic implant of claim 1, wherein the outermost coating comprises at least one selected from the group consisting of polyvinylpyrrolidone, hyaluronic acid, polylactic acid, polyethylene glycol, collagen and chitosan.

16. The cosmetic implant of claim 1, wherein the silicone gel core is formed from a reactive polydimethyl siloxane polymer having a viscosity of from 100 centipoises to 100,000 centipoises.

17. The cosmetic implant of claim 1, wherein the silicone gel core comprises at least one selected from the group consisting of Nusil MED-6342, Nusil MED-6345, Nusil MED-6350, Nusil MED-6311, Applied Silicone 40022, Applied Silicone 40135, and Applied Silicone 40008.

18. The cosmetic implant of claim 1, further comprising an intermediate coating between the silicone gel core and the outermost coating.

19. The cosmetic implant of claim 18, wherein the intermediate coating comprises a resinous silicone coating on all sides of the silicone gel core;
wherein the intermediate coating is between and adhered to both of the silicone gel core and to the outermost coating.

20. The cosmetic implant of claim 18, wherein the intermediate coating has a thickness of from 0.5 to 500 microns.

21. The cosmetic implant of claim 1, wherein the outermost coating provides an implant to implant static and kinetic coefficient of friction between 0.025 and 1.0.

22. The cosmetic implant of claim 1, wherein the outermost coating provides an implant to implant static and kinetic coefficient of friction between 0.05 and 0.6.

23. The cosmetic implant of claim 1, wherein the outermost coating provides an implant to implant static and kinetic coefficient of friction between 0.1 and 0.4.

24. The cosmetic implant of claim 18, wherein the intermediate coating has a static and kinetic coefficient of friction between 0.025 and 1.0.

25. A method of making a cosmetic implant, comprising the steps of:
forming a silicone gel core and coating the silicone gel core with an outermost continuous hydrophilic coating on all sides of the silicone gel core.

26. The method of claim 25, further comprising the step of coating the silicone gel core with an intermediate resinous silicone coating layer on all sides of the silicone gel core;
coating the intermediate coating layer with the outermost continuous hydrophilic coating on all sides of the intermediate coating layer;
wherein the intermediate coating layer is between and adhered to both of the silicone gel core and the outermost hydrophilic coating layer.

27. A method of performing cosmetic surgery on a patient, comprising the steps of:
providing a plurality of cosmetic implants, the implants comprising a silicone gel core and an outermost continuous hydrophilic coating on all sides of the silicone gel core;
making an incision in the patient to provide access to a subcutaneous pocket; and,
placing the plurality of cosmetic implants in the subcutaneous pocket.

28. The method of claim 27, further comprising the step of forming a subcutaneous pocket after making the incision.

29. A cosmetic implant, comprising a biocompatible and deformable material with a hydrophilic outer surface which is in constant contact with either human tissue or two or more cosmetic implants of same or similar composition.

30. A cosmetic implant, comprising a silicone based deformable material with a hydrophilic outer surface which is in constant contact with either human tissue or two or more cosmetic implants of same or similar composition.

31. A cosmetic implant, comprising a silicone gel with a hydrophilic outer surface which is either in constant contact with either human tissue or two or more cosmetic implants of same or similar composition.
